(19) United States
(12) Patent Application Publication
(10) Pub. No.: US 2023/0204842 A1
(43) Pub. Date: Jun. 29, 2023

(54) LIGHT DIFFUSION DEVICE AND MEDICAL EQUIPMENT USING THE SAME

(71) Applicant: FURUKAWA ELECTRIC CO., LTD., Tokyo (JP)

(72) Inventors: Keisuke TAKE, Tokyo (JP); Hideaki HASEGAWA, Tokyo (JP); Shun-ichi MATSUSHITA, Tokyo (JP)

(73) Assignee: FURUKAWA ELECTRIC CO., LTD., Tokyo (JP)

(21) Appl. No.: 18/060,640

(22) Filed: Dec. 1, 2022

(30) Foreign Application Priority Data
Dec. 2, 2021 (JP) 2021-196512

(51) Int. Cl. F21V 8/00

(52) U.S. Cl. CPC G02B 6/0008; A61N 5/067

(57) ABSTRACT

Provided is a light diffusion device capable of irradiating laser light onto a plurality of locations in a human body in a state in which a distal end portion of an optical transmission cable is placed in the human body. The light diffusion device includes an optical transmission cable including a plurality of cores, and a light refracting portion configured to refract the light emitted from each of the plurality of cores so that irradiation directions thereof are different from each other. With such a configuration, it is possible to irradiate the laser light in a plurality of directions when the distal end of the optical transmission cable is placed in the human body. Therefore, it is possible to change the location where the laser light is irradiated without extracting or re-inserting the optical transmission cable from or into the human body, thereby shortening the time required for photoimmunotherapy.

This application is based on and claims the benefit of priority from Japanese Patent Application No. 2021-196512, filed on 2 Dec. 2021, the content of which is incorporated herein by reference.

### BACKGROUND OF THE DISCLOSURE

### Field of the Disclosure

The present disclosure relates to a light diffusion device for use in the medical field and a medical equipment using the same.

### Related Art

A conventional light diffusion device has been known which includes an optical transmission cable having an optical transmission path through which light emitted from a light source is transmitted and a lens provided at a distal end portion of the optical transmission cable, and irradiates light emitted from the optical transmission cable in a predetermined direction through the lens (see, e.g., Japanese Unexamined Patent Application (Translation of PCT Application), Publication No. 2003-528347).

In photoimmunotherapy which is one therapeutic method for cancer, such a light diffusion device is used for inserting a distal end of an optical transmission cable into a human body and irradiating a drug that has been administered to the human body and reached cancer cells with laser light. In the optical diffusion device, optical fiber is often used as an optical transmission cable, and examples thereof include a cylindrical diffuser that emits light from an outer peripheral surface of the optical fiber, and a frontal diffuser that emits light from an end surface of a tip of the optical fiber.
Patent Document 1: Japanese Unexamined Patent Application (Translation of PCT Application), Publication No. 2003-528347

### SUMMARY OF THE DISCLOSURE

In photoimmunotherapy, it is necessary to irradiate laser light to a plurality of portions in the human body in a state where the distal end of the optical transmission cable of the optical diffusion device is placed in the human body or positioned in the vicinity of the tumor surface. However, in a conventional light diffusion device, in particular in a frontal diffuser, light emitted from the optical transmission cable is irradiated only in a predetermined direction. For this reason, in photoimmunotherapy, in a case of changing the location where laser light is irradiated while the distal end of the optical transmission cable is placed in the human body or positioned in the vicinity of the tumor surface, it is necessary to bend the optical transmission cable as necessary in order to adjust the irradiation direction of the laser light and re-insert the optical transmission cable into the human body. This may take a long time for photoimmunotherapy.

It is an object of the present disclosure to provide a light diffusion device capable of irradiating laser light to a plurality of locations in a human body in a state in which a distal end portion of an optical transmission cable is placed in the human body or in a state in which the distal end portion is positioned in the vicinity of a tumor surface, and a medical equipment using the same.

A light diffusion device according to an embodiment of the present disclosure includes: an optical transmission cable including a plurality of light transmission paths through which light emitted from a light source is transmitted; and a light refracting portion provided at a distal end portion of the optical transmission cable, the light refracting potion being configured to refract the light emitted from each of the plurality of light transmission paths so that irradiation directions thereof are different from each other.

Furthermore, in the light diffusion device according to an embodiment of the present disclosure, the light refracting portion is a lens having a curved surface shape in which an incident surface on which light emitted from each of the plurality of light transmission paths is incident projects toward the plurality of light transmission paths, and having a curved surface shape in which an emission surface on which incident light is emitted projects toward a light emission direction.

Furthermore, in the light diffusion device according to an embodiment of the present disclosure, the optical transmission cable includes a multicore optical fiber in which cores serving as the plurality of light transmission paths are provided in one cladding, and an irradiation direction of light is switched by switching a core that transmits the light among the cores.

Furthermore, the light diffusion device according to an embodiment of the present disclosure further includes an auxiliary light refracting portion that refracts light between the distal end portion of the optical transmission cable and the light refracting portion.

Furthermore, in the light diffusion device according to an embodiment of the present disclosure, the optical transmission cable includes a plurality of single-core optical fibers each having, in one cladding, a core as one of the plurality of light transmission paths, and an irradiation direction of light is switched by switching a single-core optical fiber that transmits the light among the plurality of single-core optical fibers.

Furthermore, in the light diffusion device according to an embodiment of the present disclosure, in the single-core optical fibers, end surfaces from which light is emitted are inclined with respect to an extending direction of the single-core optical fibers and a direction orthogonal or substantially orthogonal to the extending direction.

Furthermore, in the light diffusion device according to an embodiment of the present disclosure, the light refracting portion is attachable to and detachable from the optical transmission cable, and the light refracting portion having a different irradiation range at a portion irradiated with light is attachable to the optical transmission cable.

Furthermore, in the light diffusion device according to an embodiment of the present disclosure, the light refracting portion is configured to change a distance from the distal end portion of the optical transmission cable in a state where the light refracting portion is attached to the distal end portion of the optical transmission cable.

Furthermore, the light diffusion device according to an embodiment of the present disclosure further includes a cylindrical coupling member that couples the optical transmission cable and the light refracting portion.

Furthermore, in the light diffusion device according to an embodiment of the present disclosure, light transmitted through the optical transmission cable has a wavelength of 600 nm or more and 900 nm or less.

A medical equipment according to an embodiment of the present disclosure includes the light diffusion device.

According to an embodiment of the present disclosure, it is possible to irradiate the laser light in a plurality of directions in a state in which the distal end of the optical transmission cable is placed in the human body or in a state in which the distal end is positioned in the vicinity of the tumor surface. Therefore, it is possible to change the location where the laser light is irradiated without extracting or re-inserting the optical transmission cable from or into the human body. This makes it possible to shorten the time required for the photoimmunotherapy. Furthermore, since it is possible to irradiate the laser light on locations where it is not possible for the conventional light diffusion device to irradiate the laser light, it is possible to enlarge the application range for light irradiation in the human body. Moreover, it is possible to improve the operability of the light diffusion device.

### BRIEF DESCRIPTION OF THE DRAWINGS

FIG. 1 is a schematic diagram of a light diffusion device according to a first embodiment of the present disclosure.

FIG. 2 is a main portion cross-sectional view of the light diffusion device according to the first embodiment of the present disclosure.

FIG. 3 is a schematic diagram illustrating a path of laser light in the light diffusion device according to the first embodiment of the present disclosure.

FIGS. 4A and 4B is a schematic diagram illustrating a method of switching the irradiation direction in the light diffusion device according to the first embodiment of the present disclosure.

FIG. 5 is a main portion cross-sectional view showing another example of the optical transmission cable of the optical diffusion device according to the first embodiment of the present disclosure.

FIG. 6 is a main portion cross-sectional view showing another example of the optical transmission cable of the optical diffusion device according to the first embodiment of the present disclosure.

FIG. 7 is a main portion cross-sectional view showing another example of the optical transmission cable of the optical diffusion device according to the first embodiment of the present disclosure.

FIGS. 8A and 8B is a main portion cross-sectional view of a light diffusion device according to a second embodiment of the present disclosure.

FIG. 9 is a main portion cross-sectional view of a light diffusion device according to a third embodiment of the present disclosure.

FIG. 10 is a main portion cross-sectional view of a light diffusion device according to a fourth embodiment of the present disclosure.

### DETAILED DESCRIPTION OF THE DISCLOSURE

### First Embodiment

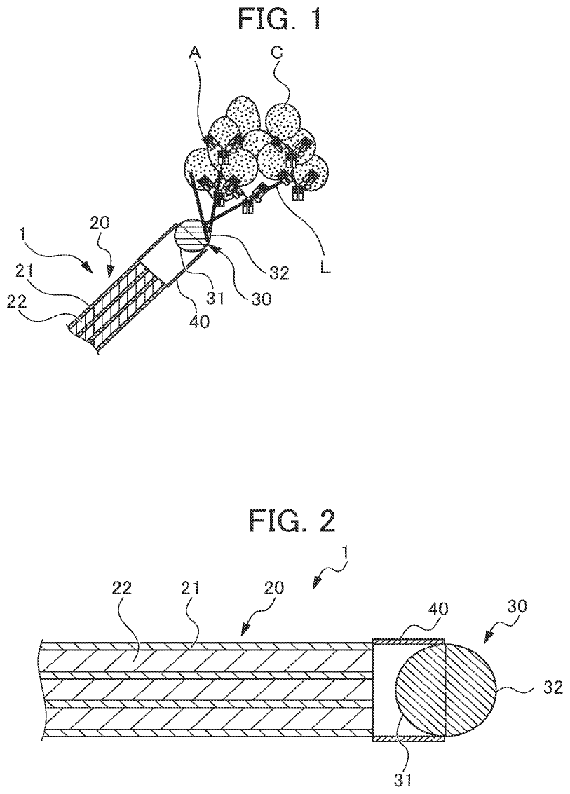

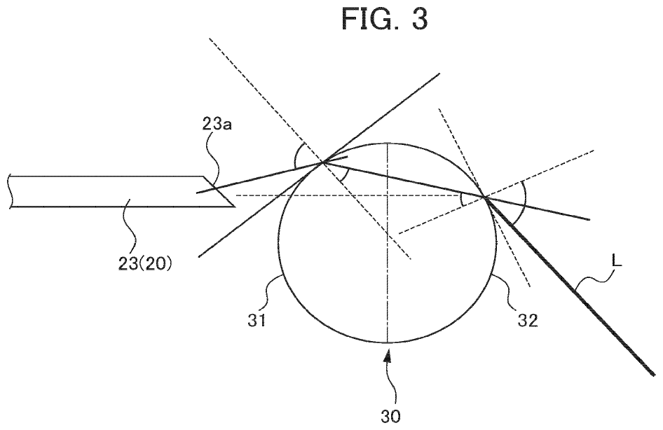

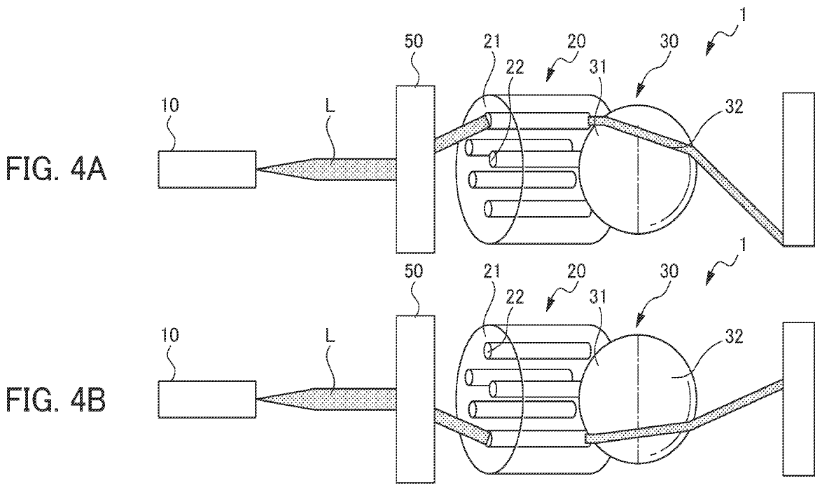

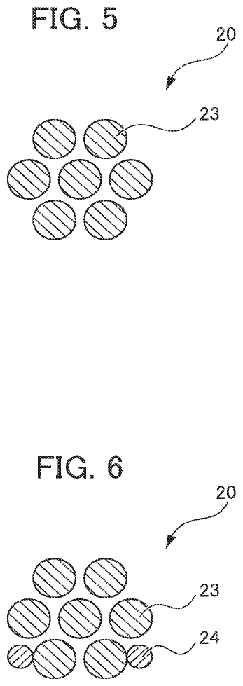

FIGS. 1 to 7 show a first embodiment of the present disclosure. FIG. 1 is a schematic diagram of a light diffusion device. FIG. 2 is a transverse cross-sectional view of the light diffusion device. FIG. 3 is a schematic diagram illustrating a path of a laser light in the light diffusion device. FIGS. 4A and 4B is a schematic diagram illustrating a method of switching an irradiation direction in the light diffusion device. FIGS. 5 to 7 are main portion cross-sectional views of other examples of an optical transmission cable.

The light diffusion device 1 of the present embodiment is mounted on a medical equipment for performing photoimmunotherapy, which is one method for treating cancer. In the photoimmunotherapy, a drug comprising an antibody A that binds to a cancer cell C and a substance that reacts with light is administered to a human body, and as shown in FIG. 1, the cancer cell C is destroyed by irradiating the drug bound to the cancer cell C with a laser light L. It is to be noted that the present disclosure is not limited to the photoimmunotherapy, and can also be used in therapeutic methods using laser light such as a photodynamic therapy.

As shown in FIGS. 2, 4A and 4B, the light diffusion device 1 includes a laser oscillator 10 as a light source for generating the laser light L, an optical transmission cable 20 for transmitting the laser light L generated in the laser oscillator 10, a light refracting portion 30 for refracting the laser light L emitted from the optical transmission cable 20, a coupling member 40 for coupling the optical transmission cable 20 and the light refracting portion 30, and an irradiation direction switching portion 50 for changing the irradiation direction of the laser light L irradiated through the light refracting portion 30 to the optical transmission cable 20.

The laser oscillator 10 includes a semiconductor laser, and generates laser oscillation by flowing electricity to the semiconductor laser to generate laser light L. The laser oscillator 10 generates red laser light L having a wavelength of 600 nm to 900 nm.

As shown in FIGS. 1, 2, 4A and 4B, the optical transmission cable 20 includes a multicore optical fiber in which a plurality of cores 22 as optical transmission paths are provided in one cladding 21. The outer diameter of the multicore optical fiber is 250 μm, for example, and the outer diameter of each core 22 is 30 μm, for example. In the optical transmission cable 20, the laser light L generated in the laser oscillator 10 is transmitted through a set core 22 among the plurality of cores 22 by the irradiation direction switching portion 50, and is emitted from the distal end portion.

The light refracting portion 30 is disposed at the distal end portion of the optical transmission cable 20, and refracts the laser light L emitted from the plurality of cores 22 at the end portion of the optical transmission cable 20 so that the irradiation directions are different from each other. The light refracting portion 30 is a lens having a curved surface shape in which an incident surface 31 on which light emitted from the distal end portion of the optical transmission cable 20 is incident projects toward the optical transmission cable 20, and having a curved surface shape in which an emission surface 32 on which incident light is emitted projects toward the light emission direction. The light refracting portion 30 is, for example, a spherical ball lens having a refractive index of 1.5 and a diameter of 1 mm. As shown in FIG. 3, the light refracting portion 30 refracts the laser light L emitted from the distal end portion of the optical transmission cable 20 at the incident surface 31 to be incident thereon, and refracts the incident laser light L at the emission surface 32 to be emitted therefrom. The light refracting portion 30 refracts the laser light L emitted from the distal end portion of the optical transmission cable 20 at a maximum angle of 90 degrees with respect to the extending direction of the core 22 on the distal end of the optical transmission cable 20.

The coupling member 40 is made of a soft resin material, for example, and has a cylindrical shape. As shown in FIGS. 1 and 2, one end of the coupling member 40 is coupled to the distal end portion of the optical transmission cable 20, and the other end of the coupling member 40 holds the light refracting portion 30 by press-fitting the light refracting portion 30.

As shown in FIGS. 4A and 4B, the irradiation direction switching portion 50 is a light selection element that causes the laser light L emitted from the laser oscillator 10 to enter the set core 22 among the plurality of cores 22 of the optical transmission cable 20. The irradiation direction switching portion 50 sets the core 22 to which the laser light L is to be incident by, for example, a switching switch. The irradiation direction switching portion 50 may cause the laser light L to enter two or more cores 22 simultaneously. Examples of the light selection element include a Mach-Zehnder interferometer type optical switch and a MEMS type optical switch.

When the light diffusion device 1 configured as described above is used for the photoimmunotherapy, the drug that has reached the cancer cell C is irradiated with the laser light L in a state in which the distal end of the optical transmission cable 20 including the light refracting portion 30 and the coupling member 40 is placed in the human body or positioned in the vicinity of the tumor surface.

Here, when the distal end of the optical transmission cable 20 is placed in a human body or positioned in the vicinity of a tumor surface, a general medical equipment such as a puncture needle, a cannula, a tube, a catheter, or an endoscope can be used together.

At this time, the laser light L generated in the laser oscillator 10 propagates through the core 22 selected from among the plurality of cores 22 of the optical transmission cable 20 by the irradiation direction switching portion 50, and is emitted from the distal end portion of the optical transmission cable 20. The laser light L emitted from the distal end portion of the optical transmission cable 20 is refracted at the curved incident surface 31 of the light refracting portion 30, and refracted at the curved emission surface 32 to be irradiated to a target location in the human body.

In addition, in a case of changing the position where the laser light L is irradiated on the human body in a state where the distal end of the optical transmission cable 20 is placed in the human body or positioned in the vicinity of the tumor surface, as shown in FIGS. 4A and 4B, the irradiation direction switching portion 50 switches the core 22 that propagates the laser light L among the plurality of cores 22 of the optical transmission cable 20 to irradiate the other position in the human body with the laser light L.

As described above, the light diffusion device 1 of the present embodiment includes the optical transmission cable 20 having the plurality of cores 22 through which the laser light L emitted from the laser oscillator 10 is transmitted, and the light refracting portion 30 provided at the distal end portion of the optical transmission cable 20 for refracting the laser light L emitted from each of the plurality of cores 22 so that the irradiation directions are different from each other.

According to the medical equipment of the present embodiment, the light diffusion device 1 is mounted.

With such a configuration, it is possible to irradiate the laser light L in a plurality of directions in a state in which the distal end of the optical transmission cable 20 is placed in the human body or in a state in which the distal end is positioned in the vicinity of the tumor surface. Therefore, it is possible to change the location where the laser light L is irradiated without extracting or re-inserting the optical transmission cable 20 from or into the human body. This makes it possible to shorten the time required for the photoimmunotherapy. Furthermore, since it is possible to irradiate the laser light L on locations where it is not possible for a conventional light diffusion device to irradiate the laser light L, it is possible to enlarge the application range for light irradiation in the human body. Moreover, it is possible to improve the operability of the light diffusion device 1.

Furthermore, it is possible that the light refracting portion 30 is a lens having a curved surface shape in which the incident surface 31 on which the laser light L emitted from the plurality of cores 22 is incident projects toward the optical transmission cable 20, and having a curved surface shape in which the emission surface 32 on which the incident laser light L is emitted projects toward the light emission direction of the laser light L.

With such a configuration, since the laser light L emitted from the distal end portion of the optical transmission cable 20 is refracted at the incident surface 31 and the emission surface 32, it is possible to increase the irradiation angle of the laser light L with respect to the extending direction of the core 22 on the distal end of the optical transmission cable 20, and it is possible to irradiate a wide range in the human body with the laser light L in a state in which the optical transmission cable 20 is placed in the human body or in a state in which the optical transmission cable 20 is positioned in the vicinity of the tumor surface.

Furthermore, it is possible that the optical transmission cable 20 has a multicore optical fiber in which a plurality of cores 22 are provided in one cladding 21, and the irradiation direction of the laser light L is switched by switching the core 22 that transmits the laser light L among the plurality of cores 22.

With such a configuration, it is possible to switch the irradiation direction of the laser light L by switching the core 22 that transmits the laser light L. Therefore, it is possible to switch the irradiation direction of the laser light L by a simple operation.

Furthermore, it is possible to include the cylindrical coupling member 40 for coupling the optical transmission cable 20 and the light refracting portion 30.

This makes it possible to couple the light refracting portion 30 to the optical transmission cable 20 with a simple configuration, thereby reducing the manufacturing cost.

The laser light L transmitted by the optical transmission cable 20 in some embodiments has a wavelength of 670 nm or more and 700 nm or less.

This makes it possible to reliably react the drug containing the antibody A in the photoimmunotherapy.

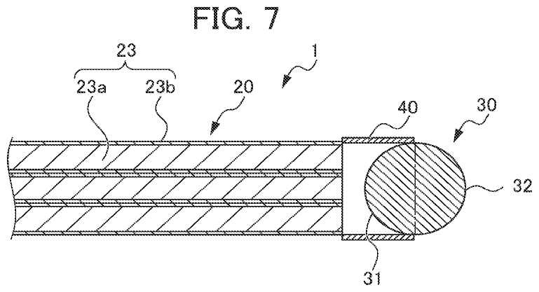

In the above embodiment, the optical transmission cable 20 has a multi-core optical fiber in which a plurality of cores 22 are provided in one cladding 21. However, the present disclosure is not limited thereto. As shown in FIG. 5, the optical transmission cable 20 may include a plurality of single-core optical fibers 23 each having a single core provided in one cladding, and may be formed by bundling a plurality of single-core optical fibers 23. In this case, it is possible to switch the irradiation direction of the laser light by a simple configuration in which the laser oscillator 10 is connected to a plurality of single-core optical fibers 23 via connectors without using the irradiation direction switching portion 50. As shown in FIG. 6, the optical transmission cable 20 may be formed by bundling a plurality of types of single-core optical fibers 23 and 24 having different outer diameters. Furthermore, as shown in FIG. 3, in the single-core optical fiber 23, the end surface 23a from which the laser light L is emitted may be inclined with respect to the extending direction of the single-core optical fiber 23 and the direction orthogonal or substantially orthogonal to the extending direction. Furthermore, as shown in FIG. 7, the optical transmission cable 20 may be configured by bundling three single-core optical fibers 23 which are aligned therein. As a representative example of the single-core optical fiber 23 constituting the optical transmission cable 20, the outer diameter of the core 23a is 200 μm, the outer diameter of the cladding 23b is 250 μm, and the relative refractive index is 1.04. The optical transmission cable 20 may be made of glass or resin.

As described above, it is possible that the optical transmission cable 20 has a plurality of single-core optical fibers 23 and 24 each having one core provided in one cladding, and that the irradiation direction of the laser light L is switched by switching the single-core optical fibers 23 and 24 for transmitting the laser light L among the plurality of single-core optical fibers 23 and 24.

With such a configuration, it is possible to switch the irradiation direction of the laser light L by switching the single-core optical fibers 23 and 24 for transmitting the laser light L, and therefore, it is possible to switch the irradiation direction of the laser light L by a simple operation.

Furthermore, in the single-core optical fiber 23, the end surface 23a from which light is emitted is inclined in some embodiments with respect to the extending direction of the single-core optical fiber 23 and the direction orthogonal or substantially orthogonal to the extending direction.

With such a configuration, it is possible to increase the incident angle of the laser light L relative to the incident surface 31 of the light refracting portion 30, and it is possible to increase the refracting angle of the laser light L in the light refracting portion 30.

### Second Embodiment

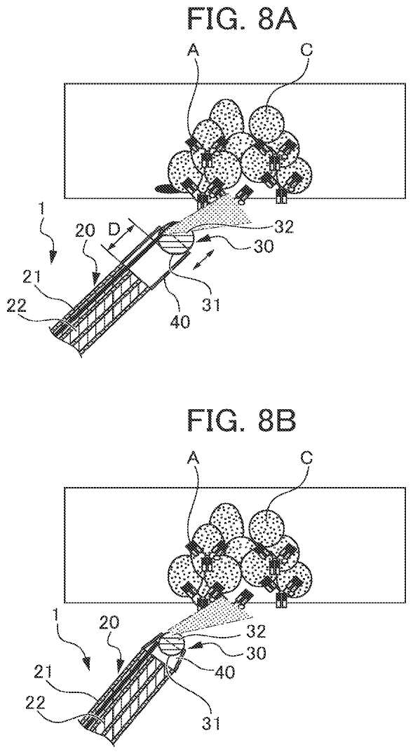

FIGS. 8A and 8B is a main portion cross-sectional view of a light diffusion device according to a second embodiment of the present disclosure. The same components as those in the above embodiment are denoted by the same reference numerals.

In the light diffusion device 1 of the present embodiment, the light refracting portion 30 is configured to be attachable to and detachable from the optical transmission cable 20. More specifically, the coupling member 40 holding the light refracting portion 30 is configured to be attachable to and detachable from the optical transmission cable 20. With such a configuration, as shown in FIGS. 8A and 8B, in the light diffusion device 1, it is possible to attach the light refracting portions 30 having different irradiation ranges of the laser light L to the optical transmission cable 20.

Furthermore, as shown in FIG. 8A, in the light diffusion device 1, it is possible to change the distance D from the distal end portion of the optical transmission cable 20 to the light refracting portion 30. With such a configuration, in the light diffusion device 1, it is possible to change the irradiation range of the laser light L and the focal distance by adjusting the distance D. The distance D can be adjusted, for example, within a range of 0 mm to 3 mm.

As described above, according to the light diffusion device 1 of the present embodiment, similarly to the first embodiment, it is possible to irradiate the laser light L in a plurality of directions in a state in which the distal end of the optical transmission cable 20 is placed in the human body or in a state in which the distal end is positioned in the vicinity of the tumor surface. Therefore, it is possible to change the location where the laser light L is irradiated without extracting or re-inserting the optical transmission cable 20 from or into the human body. This makes it possible to shorten the time required for the photoimmunotherapy. Furthermore, since it is possible to irradiate the laser light L on locations where it is not possible for the conventional light diffusion device to irradiate the laser light L, it is possible to enlarge the application range for light irradiation in the human body. Moreover, it is possible to improve the operability of the light diffusion device 1.

Furthermore, it is possible that the light refracting portion 30 is attachable to and detachable from the optical transmission cable 20, and that the light refracting portion 30 having a different irradiation range at a portion irradiated with light in the human body can be attached to the optical transmission cable 20.

With such a configuration, it is possible to use the light refracting portion 30 according to the condition of the human body which is the irradiation target of the laser light L, thereby making it possible to efficiently perform the photoimmunotherapy. Furthermore, it is possible to reduce or prevent the irradiation of laser light onto healthy cells, and it is possible to reduce invasion to the body of a patient undergoing the photoimmunotherapy.

It is possible to change the distance D from the distal end portion of the optical transmission cable 20 in a state where the light refracting portion 30 is attached to the distal end portion of the optical transmission cable 20.

With such a configuration, it is possible to change the irradiation range of the laser light L and the focal distance without attaching or detaching the light refracting portion 30 to or from the optical transmission cable 20, thereby making it possible to efficiently perform the photoimmunotherapy.

### Third Embodiment

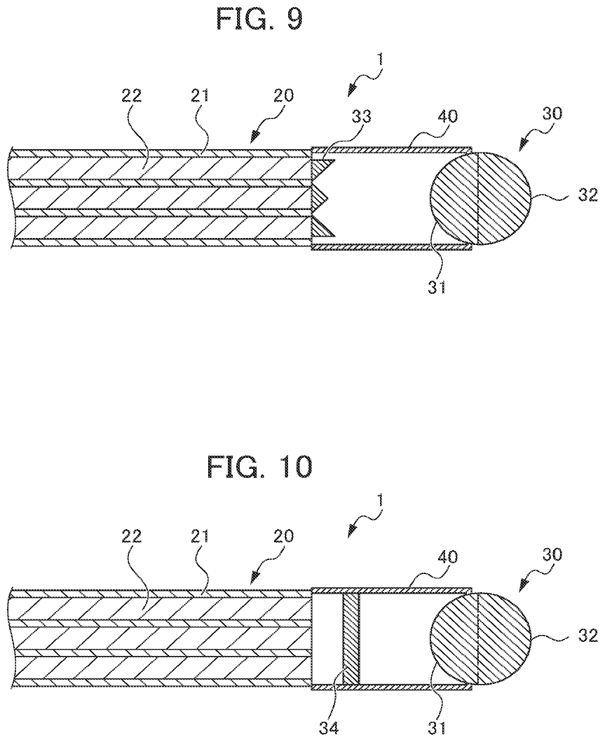

FIG. 9 is a main portion cross-sectional view of a light diffusion device according to a third embodiment of the present disclosure.

In the light diffusion device 1 of the present embodiment, the light refracting portion 30 is disposed at a distance from the distal end portion of the optical transmission cable 20, and an auxiliary light refracting portion 33 for refracting the laser light L emitted from each core and incident on the incident surface 31 of the light refracting portion 30 is attached to the end surface of each core 22 at the distal end portion of the optical transmission cable 20.

The auxiliary light refracting portion 33 is, for example, a prism in a triangular prism shape, and refracts the laser light L emitted from each core 22. The laser light L refracted in the auxiliary light refracting portion 33 is refracted twice more in the light refracting portion 30, and is irradiated to a target portion in the human body.

As described above, in the light diffusion device 1 of the present embodiment, similarly to the first embodiment, it is possible to irradiate the laser light L in a plurality of directions in a state in which the distal end of the optical transmission cable 20 is placed in the human body or in a state in which the distal end is positioned in the vicinity of the tumor surface. Therefore, it is possible to change the location where the laser light L is irradiated without extracting or re-inserting the optical transmission cable 20 from or into the human body. This makes it possible to shorten the time required for the photoimmunotherapy. Furthermore, since it is possible to irradiate the laser light L onto locations where it is not possible for a conventional light diffusion device to irradiate the laser light L, it is possible to enlarge the application range for light irradiation in the human body. Moreover, it is possible to improve the operability of the light diffusion device 1.

Furthermore, the auxiliary light refracting portion 33 for refracting the laser light L is provided between the distal end portion of the optical transmission cable 20 and the light refracting portion 30.

With such a configuration, it is possible to increase the incident angle of the laser light L relative to the incident surface 31 of the light refracting portion 30, and it is possible to increase the refracting angle of the laser light L in the light refracting portion 30.

### Fourth Embodiment

FIG. 10 is a main portion cross-sectional view of a light diffusion device according to a fourth embodiment of the present disclosure.

In the light diffusion device 1 of the present embodiment, the light refracting portion 30 is disposed at a distance from the distal end portion of the optical transmission cable 20, and a collimator lens 34 is attached between the end surface of each core 22 at the distal end portion of the optical transmission cable 20 and the light refracting portion 30 so as to make the laser light L emitted from each core 22 and incident on the incident surface 31 of the light refracting portion 30 enter the incident surface 31 of the light refracting portion 30, while maintaining the outer diameter without diffusing or focusing the laser light L.

The laser light L emitted from the end surface of each core 22 at the distal end portion of the optical transmission cable 20 is refracted by the collimator lens 34 to become collimated light, and is incident on the incident surface 31 of the light refracting portion 30.

As described above, in the light diffusion device 1 of the present embodiment, similarly to the first embodiment, it is possible to irradiate the laser light L in a plurality of directions in a state in which the distal end of the optical transmission cable 20 is placed in the human body or in a state in which the distal end is positioned in the vicinity of the tumor surface. Therefore, it is possible to change the location where the laser light L is irradiated without extracting or re-inserting the optical transmission cable 20 from or into the human body. This makes it possible to shorten the time required for the photoimmunotherapy. Furthermore, since it is possible to irradiate the laser light L onto locations where it is not possible for the conventional light diffusion device to irradiate the laser light L, it is possible to enlarge the application range for light irradiation in the human body. Moreover, it is possible to improve the operability of the light diffusion device 1.

In the above embodiment, the ball lens is shown as the light refracting portion 30 that refracts the irradiation directions of the laser lights L emitted from the plurality of cores 22 so as to be different from each other. If it is possible to refract the irradiation direction of each of the laser lights L emitted from the plurality of cores 22 so as to be different from each other, it is unnecessary to use any lens and, for example, it is possible to use a polygonal prism as a light refracting portion.

Furthermore, in the above embodiment, the spherical ball lens is shown as a lens in which the incident surface 31 on which the laser light L emitted from the plurality of cores 22 is incident has a curved surface shape protruding toward the optical transmission cable 20, and the emission surface 32 on which the incident laser light L is emitted has a curved surface shape protruding toward the emission direction of the laser light L. However, the present disclosure is not limited thereto. For example, a lens having an elliptical or cylindrical cross-sectional shape may be used as long as the lens has an incident surface on which the laser light L emitted from the plurality of cores 22 is incident having a curved surface protruding toward the optical transmission cable 20, and an emission surface on which the incident laser light L is incident having a curved surface protruding toward the emission direction of the laser light L. Furthermore, a plurality of cylindrical lenses may be used.

### EXPLANATION OF REFERENCE NUMERALS

1 light diffusion device
10 laser oscillator
20 optical transmission cable
21 cladding
22 core
23 single-core optical fiber
23a end surface
30 light refracting portion
31 incident surface
32 emission surface
33 auxiliary light refracting portion
40 coupling member
50 irradiation direction switching portion

What is claimed is:

1. A light diffusion device comprising:
an optical transmission cable including a plurality of light transmission paths through which light emitted from a light source is transmitted; and
a light refracting portion provided at a distal end portion of the optical transmission cable, the light refracting potion being configured to refract the light emitted from each of the plurality of light transmission paths so that irradiation directions thereof are different from each other.

2. The light diffusion device according to claim 1, wherein:
the light refracting portion is a lens having a curved surface shape in which an incident surface on which the light emitted from each of the plurality of light transmission paths is incident projects toward the plurality of light transmission paths, and having a curved surface shape in which an emission surface on which incident light is emitted projects toward a light emission direction.

3. The light diffusion device according to claim 1, wherein:
the optical transmission cable includes a multicore optical fiber in which cores serving as the plurality of light transmission paths are provided in one cladding, and
an irradiation direction of light is switched by switching a core that transmits the light among the cores.

4. The light diffusion device according to claim 3, further comprising:
an auxiliary light refracting portion configured to refract light between the distal end portion of the optical transmission cable and the light refracting portion.

5. The light diffusion device according to claim 1, wherein:
the optical transmission cable includes a plurality of single-core optical fibers each having, in one cladding, a core serving as one of the plurality of light transmission paths, and
an irradiation direction of light is switched by switching a single-core optical fiber that transmits the light among the plurality of single-core optical fibers.

6. The light diffusion device according to claim 5, wherein,
in the single-core optical fibers, end surfaces from which light is emitted are inclined with respect to an extending direction of the single-core optical fibers and a direction orthogonal or substantially orthogonal to the extending direction.

7. The light diffusion device according to claim 1, wherein:
the light refracting portion is attachable to and detachable from the optical transmission cable, and
the light refracting portion having a different irradiation range at a portion irradiated with light is attachable to the optical transmission cable.

8. The light diffusion device according to claim 1, wherein:
the light refracting portion is configured to change a distance from the distal end portion of the optical transmission cable in a state where the light refracting portion is attached to the distal end portion of the optical transmission cable.

9. The light diffusion device according to claim 1, further comprising:
a cylindrical coupling member that couples the optical transmission cable and the light refracting portion.

10. The light diffusion device according to claim 1, wherein light transmitted through the optical transmission cable has a wavelength of 600 nm or more and 900 nm or less.

11. A medical equipment comprising the light diffusion device according to claim 10.

12. A light diffusion device comprising:
an optical transmission cable including a plurality of light transmission paths through which light emitted from a light source is transmitted; and
a light refracting portion provided at a distal end portion of the optical transmission cable, the light refracting potion being configured to refract the light emitted from each of the plurality of light transmission paths so that irradiation directions thereof are different from each other,
wherein the light refracting portion is a lens having a curved surface shape in which an incident surface on which the light emitted from each of the plurality of light transmission paths is incident projects toward the plurality of light transmission paths, and having a curved surface shape in which an emission surface on which incident light is emitted projects toward a light emission direction.

13. The light diffusion device according to claim 12, wherein:
the optical transmission cable includes a multicore optical fiber in which cores serving as the plurality of light transmission paths are provided in one cladding, and
an irradiation direction of light is switched by switching a core that transmits the light among the cores.

14. The light diffusion device according to claim 13, further comprising an auxiliary light refracting portion configured to refract light between the distal end portion of the optical transmission cable and the light refracting portion.

15. The light diffusion device according to claim 12, wherein:
the optical transmission cable includes a plurality of single-core optical fibers each having, in one cladding, a core serving as one of the plurality of light transmission paths, and
an irradiation direction of light is switched by switching a single-core optical fiber that transmits the light among the plurality of single-core optical fibers.

16. The light diffusion device according to claim 15, wherein, in the single-core optical fibers, end surfaces from which light is emitted are inclined with respect to an extending direction of the single-core optical fibers and a direction orthogonal or substantially orthogonal to the extending direction.

17. The light diffusion device according to claim 12, wherein:
the light refracting portion is attachable to and detachable from the optical transmission cable, and
the light refracting portion having a different irradiation range at a portion irradiated with light is attachable to the optical transmission cable.

18. The light diffusion device according to claim 12, wherein:
the light refracting portion is configured to change a distance from the distal end portion of the optical transmission cable in a state where the light refracting portion is attached to the distal end portion of the optical transmission cable.

19. The light diffusion device according to claim 12, further comprising:
a cylindrical coupling member that couples the optical transmission cable and the light refracting portion.

20. The light diffusion device according to claim 12, wherein light transmitted through the optical transmission cable has a wavelength of 600 nm or more and 900 nm or less.
